(19) United States
(12) Patent Application Publication
(10) Pub. No.: US 2013/0245569 A1
(43) Pub. Date: Sep. 19, 2013

(54) Accessory Device for Inner Ear Drug Delivery

(71) Applicant: MED-EL ELEKTROMEDIZINISCHE GERAETE GMBH, Innsbruck (AT)

(72) Inventors: Claude Jolly, Innsbruck (AT); Anandhan Dhanasingh, Innsbruck (AT); Pavel Mistrik, Pardubice (CZ); Soeren Schilp, Innsbruck-Igls (AT); Roland Hessler, Innsbruck (AT); Kenneth Mugridge, Innsbruck (AT)

(73) Assignee: MED-EL ELEKTROMEDIZINISCHE GERAETE GMBH, Innsbruck (AT)

(21) Appl. No.: 13/784,973

(22) Filed: Mar. 5, 2013

Related U.S. Application Data
(60) Provisional application No. 61/611,128, filed on Mar. 15, 2012.

(51) Int. Cl. A61M 31/00; A61F 11/00

(52) U.S. Cl. CPC A61M 31/00; A61F 11/00
USPC 604/265; 604/272

(57) ABSTRACT

An implantable drug delivery accessory is described. A middle ear guide member has a hollow interior enclosed by a non-permeable outer surface. A proximal end of the middle ear guide member is fixed to the tympanic membrane of an implanted patient. A distal end of the middle ear guide member penetrates through a fluid-tight sealed opening into the inner ear. An inner ear holding member has a proximal end connected to the distal end of the middle ear guide member at the sealed opening and a closed distal end terminating the inner ear holding member some predetermined distance within the cochlea. A hollow interior of the inner ear holding member contains a replaceable drug delivery member which stores a therapeutic drug. A semi-permeable outer surface of the inner ear holding member is connected to a cochlear implant electrode and adapted to release therapeutic drug from the hollow interior into adjacent tissue. The drug delivery member is removable and replaceable via the ear canal of the implanted patient through the proximal end of the middle ear.

This application claims priority from U.S. Provisional Patent Application 61/611,128, filed Mar. 15, 2013, which is incorporated herein by reference.

### TECHNICAL FIELD

The present invention relates to medical implants, and more specifically to an accessory device for a drug delivery device for the inner ear.

### BACKGROUND ART

A human ear normally transmits sounds such as speech sounds as shown in FIG. 1 through the outer ear 101 to the tympanic membrane (eardrum) 102, which moves the bones of the middle ear 103 (malleus, incus, and stapes) that vibrate the oval window membrane of the cochlea 104. The cochlea 104 is a long narrow duct wound spirally about its axis for approximately two and three quarters turns. It includes three chambers along its length: an upper chamber known as the scala vestibuli, a middle chamber known as the scala media, and a lower chamber known as the scala tympani. The cochlea 104 forms an upright spiraling cone with a center called the modiolus where the axons of the auditory nerve 113 reside. These axons project in one direction to the cochlear nucleus in the brainstem and they project in the other direction to the spiral ganglion cells and neural processes peripheral to the cells (hereinafter called peripheral processes) in the cochlea. In response to received sounds transmitted by the middle ear 103, sensory hair cells in the cochlea 104 function as transducers to convert mechanical motion and energy into electrical discharges in the auditory nerve 113. These discharges are conveyed to the cochlear nucleus and patterns of induced neural activity in the nucleus are then conveyed to other structures in the brain for further auditory processing and perception.

Hearing is impaired when there are problems in the ability to transmit sound from the external to the inner ears or problems in the transducer function within the inner ear. To improve impaired hearing, auditory prostheses have been developed. For example, when the impairment is related to the operation of the middle ear 103, a conventional hearing aid may be used to provide acoustic stimulation to the auditory system in the form of amplified sound. Or when the impairment is associated with the transducer function in the cochlea 104, a cochlear implant system can electrically stimulate auditory neural tissue with small currents delivered by multiple electrode contacts distributed along at least a part of the cochlear length (spiral). Arrays of such electrode contacts normally are inserted into the scala tympani. Alternatively, groups of auditory nerve axons can be stimulated with electrode contacts placed within the modiolus, or auditory structures in the brain can be stimulated with electrode contacts placed on or within the structures, for example, on or within the cochlear nucleus.

FIG. 1 also shows some components of a typical cochlear implant system. The system includes an external microphone that provides an audio signal input to an external signal processor 111 which implements a specific signal processing strategy to derive patterns of electrical stimuli from the audio signal input and converts these patterns into a digital data format, such as a sequence of data frames, for transmission from an external transmitter coil 107 to a receiver coil of an implanted stimulator module 108. Besides receiving the processed audio information, the stimulator module 108 also performs additional signal processing such as error correction, pulse formation, etc., and produces electrical stimuli (based on the received data signals) that are sent through an electrode lead 109 to electrode contacts 110 in an implanted intracochlear electrode 112 to provide selective electrical stimulation of cochlear neural tissue associated with the auditory nerve 113.

The efficiency of a cochlear implant depends significantly on the functional state of the auditory nerve and its associated neural tissue. However, the auditory nerve tissue degenerates both chronically over time as a result of prolonged sensorineural deafness and acutely after CI surgery (due to electrode insertion trauma). Preventing auditory nerve degeneration currently involves an intraoperative pharmaceutical treatment during cochlear implantation (e.g., a single dose of anti-inflammatory drugs such as corticosteroids). However, it is known from animal studies that pharmacological treatment is much more beneficial when delivered for a longer period of time using drug delivery devices such as osmotic pumps, catheters or hydrogels. Still, many devices like these have only a limited volume drug reservoir, which restricts the delivery period to several weeks, after which the state of the auditory nerve tissue again starts to deteriorate. So the preferential long-term pharmacological treatment in patients (months to years) requires re-implantation of a refilled device. Such re-implantation poses risks to the patient health due to the possibility of infection.

Conventional drug delivery to the inner ear typically is based on deposition of the drug at the round window and relying on diffusion of the drug through the round window to reach targeted cells. This may be accomplished by flooding the middle ear cavity with a liquid form drug, or by applying a soaked sponge at or near the round window, for example, through an opening in the tympanic membrane. But a diffusion process through the round window is not very predictable or reliable, among other things, the permeability of the round window varies between patients. Another alternative is a single intra-operative drug application into the inner ear cavity through the surgical opening in the round window or cochleostomy before the insertion of the CI electrode array. But this approach allows only a single time-point application of the drug.

U.S. Patent Publication 2007/0077270 (incorporated herein by reference) describes a drug delivery device that may be incorporated into a cochlear implant electrode. Following surgery, the device allows prolonged delivery over time of therapeutic drugs into the tissues adjacent to the electrode. But the device increases the bulk of the implant electrode, which ironically increases the insertion trauma.

U.S. Patent Publication 2009/0248156 (incorporated herein by reference) describes piercing the tympanic membrane with a narrow tube that conducts sound from the ear canal and delivers it near to the oval window or the round window. This device acts as an auditory prosthesis to bypass any defective structures in the middle ear to directly acoustically stimulate the oval window or the round window. There is no suggestion of any application to neural tissues within the inner ear.

### SUMMARY

Embodiments of the present invention are directed to an implantable drug delivery accessory. A middle ear guide member has a hollow interior enclosed by a non-permeable outer surface. A proximal end of the middle ear guide member is fixed to the tympanic membrane of an implanted patient. A distal end of the middle ear guide member penetrates through a fluid-tight sealed opening into the inner ear. An inner ear holding member has a proximal end connected to the distal end of the middle ear guide member at the sealed opening and a closed distal end terminating the inner ear holding member some predetermined distance within the cochlea. A hollow interior of the inner ear holding member contains a replaceable drug delivery member which stores a therapeutic drug. A semi-permeable outer surface of the inner ear holding member is connected to a cochlear implant electrode and adapted to release therapeutic drug from the hollow interior into adjacent tissue. The drug delivery member can be removed and replaced via the ear canal of the implanted patient through the proximal end of the middle ear.

The accessory device may further include a guide wire enclosed within the hollow interior of the middle ear guide member having a distal end connected to the drug delivery member for surgical handling of drug delivery member. At least one of the outer surfaces may be covered by an anti-microbial coating. At least one of the members may be made of a biocompatible metal. The proximal end of the middle ear guide may be fixed to the tympanic membrane using a grommet

Embodiments of the present invention also include a method of surgically implanting a drug delivery accessory in a patient. A closed distal end of an inner ear holding member is inserted some predetermined distance through an opening into the cochlea of the patient. An outer surface of the inner ear holding member is connected to a cochlear implant electrode and includes a semi-permeable diffusion portion adapted to release into adjacent tissue a therapeutic drug from a drug delivery member within the inner ear holding member. The opening is sealed around a proximal end of the inner ear holding member and distal end of a middle ear guide member. A proximal end of the middle ear guide member is fixed to the tympanic membrane of the patient. The drug delivery member is removable and replaceable via the ear canal of the implanted patient through the proximal end of the middle ear.

In further such methods, the middle ear guide member may enclose a guide wire having a distal end connected to the drug delivery member for surgical handling of the drug delivery member. At least one of the middle ear guide member and the inner ear holding member may be covered by an anti-microbial coating. At least one of the middle ear guide member and the inner ear holding member may be made of a biocompatible metal. The proximal end of the middle ear guide may be fixed to the tympanic membrane using a grommet

### BRIEF DESCRIPTION OF THE DRAWINGS

FIG. 1 shows anatomical structures and system components in a human ear having a cochlear implant system.

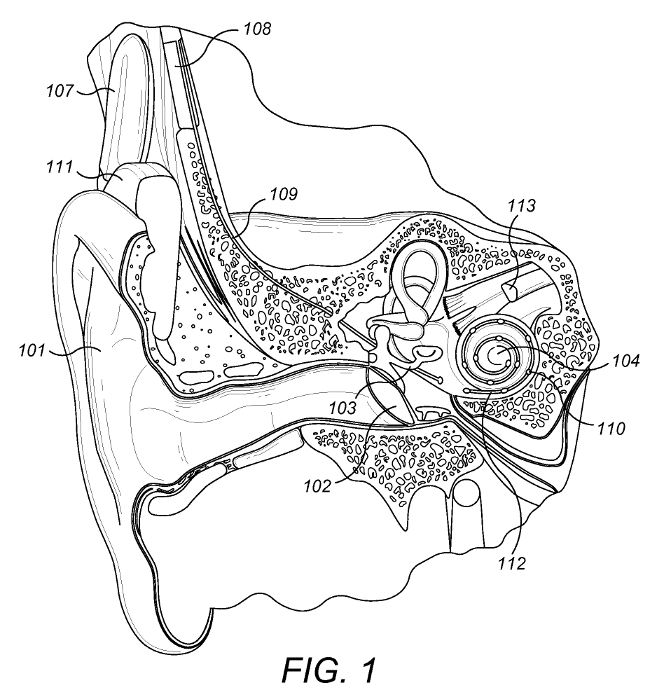

FIG. 2A-B show embodiments of an implantable drug delivery accessory having an inner ear holding member and a middle ear access.

FIG. 3A-B show embodiments of a drug delivery accessory where the drug delivery device is split into multiple drug delivery pieces.

FIG. 4 shows an embodiment of a drug delivery accessory which is connected to a cochlear electrode array.

FIG. 5 shows an embodiment of a drug delivery accessory which extends up to the temporal bone.

FIG. 6 shows an embodiment of a drug delivery accessory that extends from the tympanic membrane.

FIG. 7 shows another embodiment of a drug delivery accessory that extends from the tympanic membrane.

### DETAILED DESCRIPTION

Various embodiments of the present invention are directed to a permanently implantable implantable drug delivery accessory device which contains a replaceable drug delivery member that releases a therapeutic drug over time into adjacent tissues. Once the drug is depleted, the drug delivery member can be removed and replaced with a fresh one without disturbing the accessory device embedded in the patient's tissues.

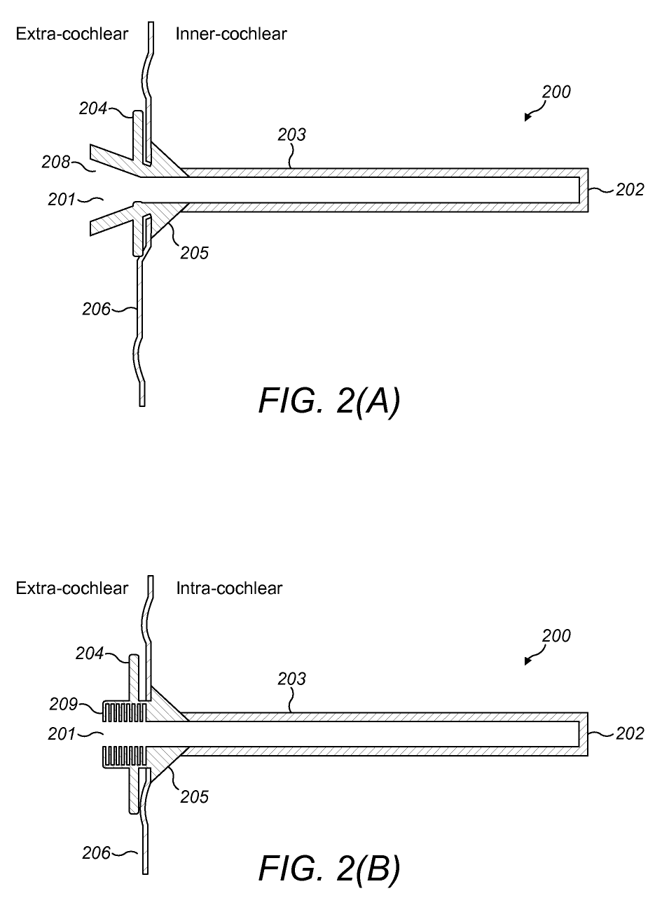

FIG. 2A shows an example of one embodiment of an implantable drug delivery accessory 200 having an intra-cochlear inner ear holding member 201 and an extra-cochlear middle ear access 201. The drug delivery accessory 200 penetrates into the cochlea through a sealed opening (e.g., through the round window or cochleostomy opening) in the cochlear wall 206. The embodiment in FIG. 2A uses a sealing arrangement of a double cork 205 and insertion stopper 204 where the smaller cork goes inside the opening in the cochlear wall 206 and fixes the drug delivery accessory 200 within the cochlea. The insertion stopper 204 limits the insertion depth The proximal end of the drug delivery accessory 200 has a cone shaped access opening 208 for inserting a drug delivery device into the inner ear holding member 201. FIG. 2B shows an embodiment of a drug delivery accessory 200 where the access opening 209 uses a threaded arrangement for better fixation of the drug delivery device within the drug delivery accessory 200.

The outer surface 203 of the inner ear holding member 201 is semi-permeable (e.g., a semi-permeable membrane or a polymeric wire mesh) to allow therapeutic drug to be released over time from the inner drug delivery member into the adjacent tissues. The inner ear holding member 201 may be flexible enough to insert into the spiral turns of the cochlea, either just into the first turn, or as far as up to the alt turn of the cochlear spiral.

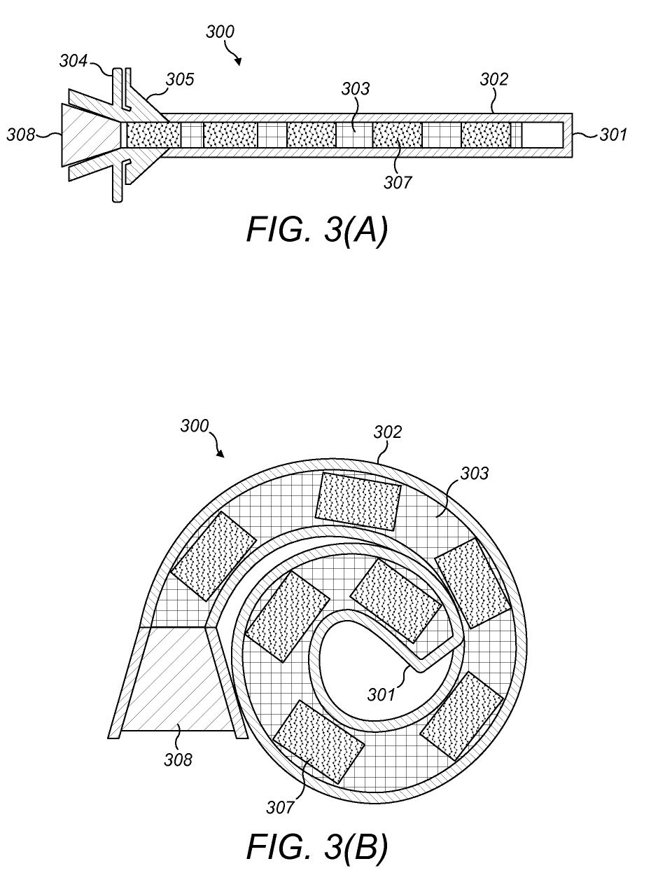

FIG. 3A-B shows an embodiment of a drug delivery accessory 300 where the drug delivery device is split into multiple drug delivery pieces 307 which are held together within the inner ear holding member 301 by a secondary mesh 303. Splitting the drug delivery device into multiple drug delivery pieces 307 may allow better fitting into the curved spiral-shaped cochlea. The proximal end of the drug delivery accessory 300 is fixed in a sealed opening in the cochlear wall by a cork 305 and insertion stopper 304 with a removable cone-shaped access cork 308 sealing the opening in the middle ear.

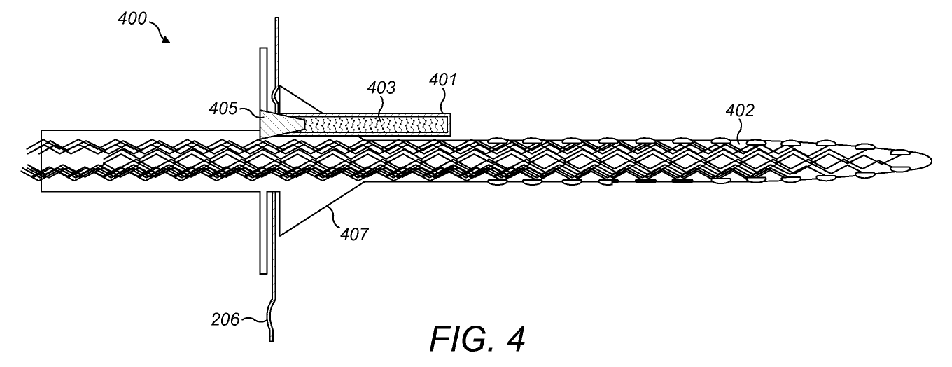

FIG. 4 shows an embodiment of a drug delivery accessory 400 which is connected to a cochlear electrode array 402 at the basal end immediately after the insertion stopper 407. The portion of the outer surface of the inner ear holding member 401 that is not in contact with the electrode array 402 is semi-permeable to allow diffusion of the therapeutic drug from the enclosed drug delivery member 403.

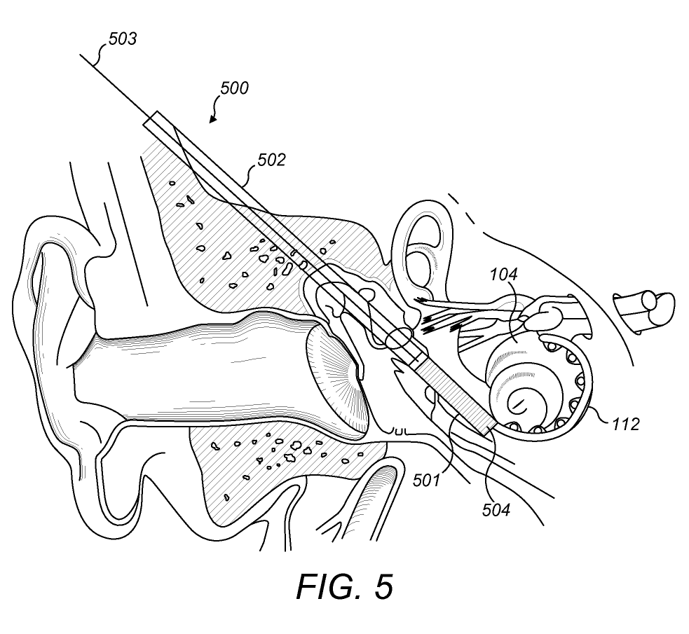

FIG. 5 shows an embodiment of a drug delivery accessory 500 which extends up to the temporal bone. A guide wire 503 extends through an outer guide member 502 and attaches at its distal end to a drug delivery member 504 within the inner ear holding member 501. The guide wire 503 allows handling, insertion and replacement of the drug delivery member 504 within the inner ear holding member 501.

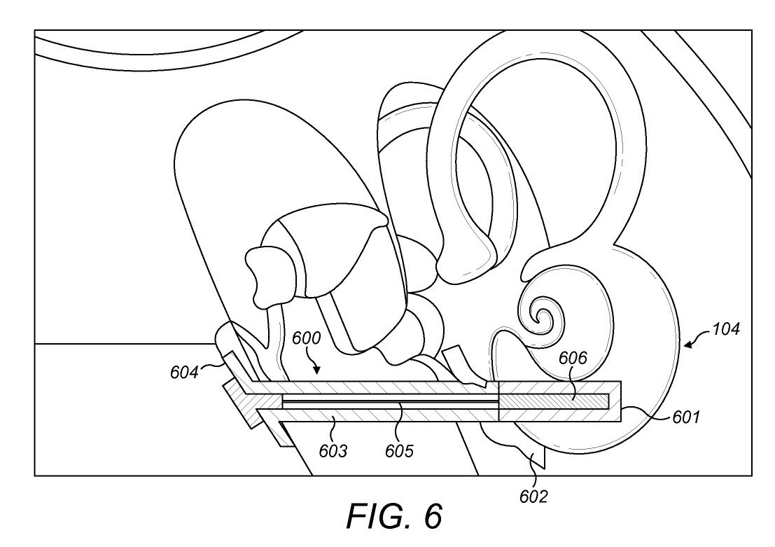

FIG. 6 shows an embodiment of an implantable drug delivery accessory 600 that extends from the tympanic membrane to penetrate into the cochlea 104. A middle ear guide member 603 has a hollow interior. A tympanic grommet 604 fixes the proximal end of the middle ear guide member 603 to the tympanic membrane, and the distal end of the middle ear guide member 603 penetrates through a fluid-tight sealed opening 602 (e.g., round window or cochleostomy entrance) into the cochlea 104. An inner ear holding member 601 has a proximal end connected to the distal end of the middle ear guide member 603 at the sealed opening 602 and a closed distal end terminating the inner ear holding member some predetermined distance within the cochlea 104. The sealed opening 602 entry point into the cochlea 104 is closed tight to prevent any leakage of the cochlear fluid out of the cochlea 104 and to prevent infection from the middle ear region. The sealed opening 602 can be accomplished based on known techniques such as a cork-type arrangement, threaded through-opening, fibrin glue etc.

A hollow interior of the inner ear holding member 601 contains a replaceable drug delivery member 606 which stores a therapeutic drug which may include cells producing proteins and/or other biomolecules for the regeneration of the spiral ganglion cells. A semi-permeable outer surface of the inner ear holding member 601 (e.g., a semi-permeable membrane or a polymeric wire mesh) is adapted to release the therapeutic drug from the drug delivery member 606 into adjacent tissue within the cochlea 104. In some embodiments, the inner ear holding member 601 may be made of a biocompatible metal with an arrangement of holes in its outer surface for releasing the therapeutic drug from the drug delivery member 606. Only the length of the inner ear holding member 601 that is within the cochlea 104 is semi-permeable and exposed to cochlear fluid. The outer surface of the middle ear guide member 603 is non-permeable.

The drug delivery member 606 is removable and replaceable by a guide wire 605 enclosed within the middle ear guide member 603 via the ear canal of the implanted patient through the proximal end of the middle ear. The distal end of the guide wire 605 is connected to the drug delivery device 606. The proximal end of the guide wire 605 provides for holding and control, and makes a firm connection with the access point of the fixed proximal end of the middle ear guide member 603 at the tympanic membrane, for example, by cork-type fitting, threaded fitting, magnetic coupling, etc.

The inner ear holding member 601 may be adapted to remain permanently within the cochlea 104. The tympanic grommet 604 allows access through the ear canal and the tympanic membrane to the guide wire 605 for insertion and/or replacement of the drug delivery member 606, which can be done by a standalone procedure or in combination with the cochlear implantation surgery. When the therapeutic drug in the drug delivery device 606 is depleted, the drug delivery member 606 is removed from the inner holding member 601 (which remains fixed within the cochlea 104) by pulling the guide wire 605 and replaced with a new one without needing any surgical technique.

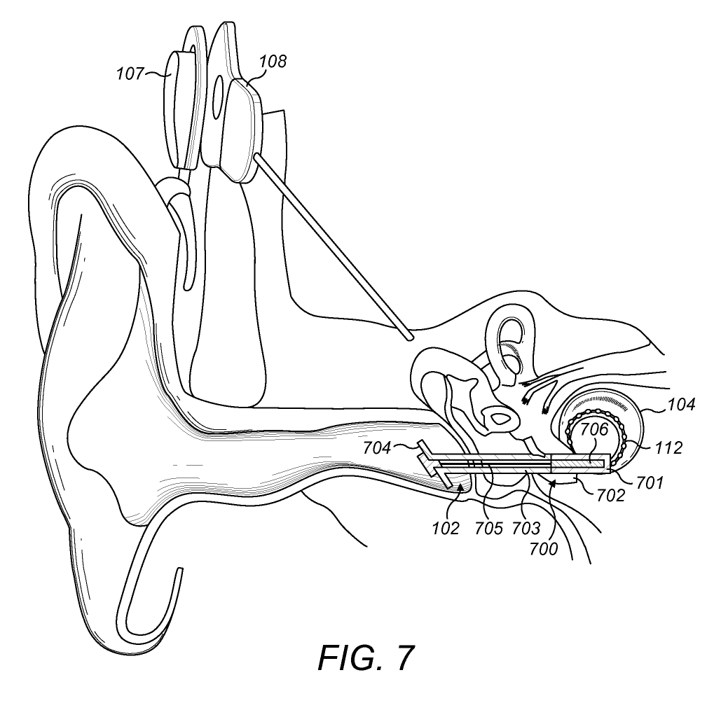

FIG. 7 shows another embodiment of an implantable drug delivery accessory 700 that extends from the tympanic membrane to penetrate into the cochlea 104, where the outer surface of the inner ear holding member 701 is connected to a cochlear implant electrode 112. The remaining exposed portion of the outer surface of the inner ear holding member 701 adapted to release therapeutic drug from the drug delivery member 706 into adjacent tissue as in the embodiment shown in FIG. 6.

Although various exemplary embodiments of the invention have been disclosed, it should be apparent to those skilled in the art that various changes and modifications can be made which will achieve some of the advantages of the invention without departing from the true scope of the invention.

What is claimed is:

1. An implantable drug delivery accessory comprising:
a middle ear guide member having:
a hollow interior,
a non-permeable outer surface enclosing the hollow interior,
a proximal end fixed to the tympanic membrane of an implanted patient, and
a distal end penetrating through a fluid-tight sealed opening into the inner ear; and
an inner ear holding member having:
a proximal end connected to the distal end of the middle ear guide member at the sealed opening,
a closed distal end terminating the inner ear holding member some predetermined distance within the cochlea,
a hollow interior for containing a replaceable drug delivery member which stores a therapeutic drug,
a semi-permeable outer surface connected to a cochlear implant electrode and adapted to release therapeutic drug from the hollow interior into adjacent tissue,
whereby the drug delivery member is removable and replaceable via the ear canal of the implanted patient through the proximal end of the middle ear.

2. An accessory according to claim 1, further comprising:
a guide wire enclosed within the hollow interior of the middle ear guide member having a distal end connected to the drug delivery member for surgical handling of drug delivery member.

3. An accessory according to claim 1, wherein at least one of the outer surfaces is covered by an anti-microbial coating.

4. An accessory according to claim 1, wherein at least one of the members is made of a biocompatible metal.

5. An accessory according to claim 1, wherein the proximal end of the middle ear guide is fixed to the tympanic membrane using a grommet.

6. A method of surgically implanting a drug delivery accessory in a patient, the method comprising:
inserting a closed distal end of an inner ear holding member some predetermined distance through an opening into the cochlea of the patient;
connecting an outer surface of the inner ear holding member to a cochlear implant electrode, wherein the outer surface includes a semi-permeable diffusion portion adapted to release into adjacent tissue a therapeutic drug from a drug delivery member within the inner ear holding member;
sealing the opening around a proximal end of the inner ear holding member and distal end of a middle ear guide member; and
fixing a proximal end of the middle ear guide member to the tympanic membrane of the patient;
whereby the drug delivery member is removable and replaceable via the ear canal of the implanted patient through the proximal end of the middle ear.

7. A method according to claim 6, wherein the middle ear guide member encloses a guide wire having a distal end connected to the drug delivery member for surgical handling of drug delivery member.

8. A method according to claim 6, wherein at least one of the middle ear guide member and the inner ear holding member is covered by an anti-microbial coating.

9. A method according to claim 6, wherein at least one of the middle ear guide member and the inner ear holding member is made of a biocompatible metal.

10. A method according to claim 6, wherein the proximal end of the middle ear guide is fixed to the tympanic membrane using a grommet.
